(19) United States
(12) Patent Application Publication
(10) Pub. No.: US 2008/0214669 A1
(43) Pub. Date: Sep. 4, 2008

(54) Composition and Method For Treating Hyperpigmented Skin

(75) Inventors: Nava Dayan, Fair Lawn, NJ (US); Jed A. Riemer, Scarsdale, NY (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LIPO CHEMICALS INC., Paterson, NJ (US)

(21) Appl. No.: 11/793,966

(22) PCT Filed: Dec. 20, 2005
(86) PCT No.: PCT/US05/46175

§ 371 Date: Nov. 13, 2007

Related U.S. Application Data
(60) Provisional application No. 60/643,797, filed on Jan. 14, 2005.
(60) Provisional application No. 60/652,303, filed on Feb. 11, 2005.

(51) Int. Cl. A61K 31/192; A61P 17/00

(52) U.S. Cl. 514/570

(57) ABSTRACT

An improved cosmetic and dermatological composition and a method for treating hyperpigmented skin is disclosed. The composition demonstrates an enhanced ability to lighten mammalian skin color, and is nontoxic and nonirritating. The composition comprises a hydroxycinnamic acid or a methoxycinnamic acid dissolved in a compound having one or more hydroxy groups, like a monoC₁₋₄alkyl ether of an ethylene glycol or a monoC₁₋₄alkyl ether of a propylene glycol oligomer, and/or a silicone fluid.

### CROSS REFERENCE TO RELATED APPLICATIONS

This application claims the benefit of U.S. provisional patent application Ser. No. 60/652,303, filed Feb. 11, 2005, and U.S. provisional patent application Ser. No. 60/643,797, filed Jan. 14, 2005.

### FIELD OF THE INVENTION

The present invention relates to cosmetic and dermatological compositions capable of treating hyperpigmented skin. More particularly, the present invention relates to compositions having an enhanced ability to lighten the color of mammalian skin. The compositions comprise a hydroxycinnamic acid or a methoxycinnamic acid dissolved in an organic compound having one or more hydroxy groups, for example, a monoC₁₋₄alkyl ether of an ethylene glycol oligomer or a monoC₁₋₄alkyl propylene glycol oligomer, and/or a silicone fluid.

### BACKGROUND OF THE INVENTION

The administration of an active ingredient frequently is limited by natural barriers which pre-vent adequate introduction of the active ingredient to the desired target site, typically because the barrier is not sufficiently permeable to the active ingredient. For topically applied compounds, the natural barrier is the upper layer of the skin.

Human skin consists of two compartments, i.e., a deep compartment, termed the dermis, and a superficial compartment, termed the epidermis. The dermis supports and nourishes the epidermis. The epidermis is in contact with the external environment, and its role is to protect the body against dehydration and external attack, whether chemical, mechanical, physical, or infectious in nature. The human epidermis primarily is composed of several types of cells, e.g., the keratinocytes, the melanocytes, and the Langerhans cells. Each type of cell contributes to the essential role played by the skin by virtue of its intrinsic functions.

The epidermis of mammals can exhibit hyperpigmentation, e.g., a skin color that is esthetically too dark or uneven in tone. The American Academy of Dermatology has estimated that 5 to 6 million Americans will suffer from a skin pigmentation condition at some point of their lives. It also has been reported that melasma occurs in 50% to 70% of pregnant females, and that about 90% of light-skinned Caucasians over 60 years old develop liver spots. Hyperpigmentation can have a profound negative impact on the social, emotional, and psychological well-being of an individual.

Hyperpigmentation, or an abnormally increased pigmentation or melanin deposition, can be attributed to different etiologies, including local hyperpigmentation from drug use (e.g., calcium antagonists), cyanic melasma, senile melasma, vitiglio, adverse sequelae following sclerotherapy, or postinflammatory or traumatic responses. Other local hyperpigmentations can occur during pregnancy (known as gravidic chloasma), after estro-progestative contraception, by photosensitization, or by postlesional cicatrization.

For example, hyperpigmentation includes age spots, “solar lentigo,” or “liver spots,” mainly resulting from excessive sun exposure, and which are common on the hands, face, forearms; melasma in pregnant women (i.e., “the mask of pregnancy”) or in women taking oral contraceptives; drug-induced or postinflammatory skin darkening; and disease-related skin darkening, such as in Addison's disease. Hyperpigmentation also can result from a cumulative sun exposure throughout life, which leads to age spots or “sun-induced freckles.” Hyperpigmentation further can be attributed to an intrinsic genetic profile, e.g., individuals having melanocytes that secrete melanin in the absence of ultraviolet (UV) exposures.

A highly-pigmented skin also may be considered an unaesthetic to individuals in various ethnic groups, and who therefore wish to reduce skin color. For example, the first depigmenting cream appeared in Korea decades ago as the result of an esthetic desire of some Asian females to have a pale facial complexion. These initial depigmenting creams contained a mercury compound, whose action was based on the substitution of copper, an essential cofactor of the tyrosinase enzyme in the pathway that generates melanin from tyrosine. Mercurials have since been banned because of their neurotoxicity.

Skin coloration is directly related to melanin formation. Melanin is synthesized in melanocytes found in the epidermal basal layer between proliferated keratinocytes before terminal differentiation. The more melanin that is produced, the darker the skin color. Melanin formation in turn is directly related to the action of tyrosinase on tyrosine and cysteine. When tyrosinase activity is inhibited, the enzymatic conversion of tyrosine and cysteine to melanin is reduced. The end result is a preventative or curative skin lightening because the production of melanin is reduced or precluded.

Current treatments for hyperpigmentation include using a sunscreen, which prevents tyrosinase activity; melanocyte toxicity due to an application of a compound like hydroquinone; exfoliation; inhibition of melanocyte transfer to keratinocytes using a compound like niacinamide; inhibition of tyrosinase; and combinations of such treatments.

Previously-used skin depigmenting agents included peroxides, such as hydrogen peroxide, zinc peroxide, sodium peroxide, benzoyl peroxide, and the like. However, peroxide activity often is coupled with adverse side effects. Several presently used natural compounds partially inhibit melanin synthesis and/or tyrosinase activity, for example, glucosamines, galactosamines, mannosamines, and some plant extracts, whose action has been correlated to blocking of free radicals which are the true stimulating factors of melanogenesis. Plant extracts also suffer from the disadvantages of instability, a lack of standardized product, and low efficacy. Antioxidants, such as vitamin C and E and esters thereof, also exhibit moderate depigmenting activity, with partial inhibition of melanogenesis. However, such antioxidants typically are not sufficiently efficacious. Azelaic acid also has been used as a depigmenting agent because it demonstrates a competitive inhibition of tyrosinase and of the DNA synthesis within melanocytes.

Tyrosinase inhibitors have become increasingly important in cosmetic and medicinal products in the treatment of hyperpigmentation. A few anti-melanogenic reagents, such as monobenzone and hydroquinone, also are clinically useful. Hydroquinone is a tyrosinase substrate with antagonist and competitive action on tyrosine.

Hydroquinone and its derivatives, such as the monomethyl ether of hydroquinone and arbutin, are the most common depigmenting agents used in topical compositions. Prescription skin lightening compositions may contain 3% to 5%, by weight, hydroquinone. However, the dosage typically is limited to a concentration of 2%, by weight, because hydroquinone is unstable and irritating, and is cytotoxic to melanocytes, with indications of localized granular hyperpigmentation and formation of elastosis, as well as the occurrence of vitiligo after long-term use. See WO 01/17497, incorporated herein by reference.

Another tyrosinase inhibitor is kojic acid, which is unstable, exhibits weak mutagenicity, and is a skin sensitizer and irritant. Ferulic acid is a relatively nonefficacious tyrosinase inhibitor. Glabridin (licorice extract) suffers from instability and purity concerns, and a high cost. Arbutin suffers from a high cost. These tyrosinase inhibitors also suffer from a relatively low efficacy because of a poor skin permeation to the target site of action, i.e., the melanocytes.

A number of skin lightening compounds also have been partially or completely banned because of toxicity and environmental concerns. For example, mercury, hydroquinone, hormonal preparations, and oxidizing agents have been banned as skin-lightening agents. Kojic acid is the leading active agent used today for skin lightening. Kojic acid is fungi derived and has disadvantages, such as instability, e.g., undergoes photodegradation with time that reduces efficacy; a tendency to turn yellow to brown in formulations over time; mutagenicity and tumor promotion; irritation with sensitization potential; and a provocation of skin contact allergies. These disadvantages have led to a partial ban of kojic acid as a skin-lightening agent.

The present invention is directed to using an effective tyrosinase inhibitor that overcomes the disadvantages of prior tyrosinase inhibitors in the treatment of hyperpigmentation. The tyrosinase inhibitor must be effective, nontoxic, and stable, and capable of application from a delivery system that permits an effective amount of the tyrosinase inhibitor to penetrate the skin and reach the target site for tyrosinase inhibition.

Hydroxycinnamic acid is an antioxidant phenolic compound, which is common in plants, mainly as a component of cell walls. Hydroxycinnamic acid is an antioxidant having radical scavenging activity, and also demonstrates chemoprotective properties (H. K. Kuzaki et al., J. Agric. Food Chem., 50, 2161-68 (2002)). Compared with other tyrosinase inhibitors, p-hydroxycinnamic acid, and related hydroxycinnamic acids and ethers thereof, herein collectively termed “HCAs,” are nontoxic, colorless, and odorless, and, therefore, are excellent candidates for use as skin-lightening agents.

Tyrosinase, an enzyme present in melanosome granules within the melanocytes, catalyzes the rate-limiting step of melanin biosynthesis (S. H. Pomerantz et al., J. Clin. Invest., 55, 1127-31 (1975)). Melanin production and deposition are responsible for the variations in human pigmentation and skin tone among different racial groups. Tyrosinase inhibitors are used to even skin tone and treat pigmentation disorders, such as age spots and pregnancy mask. HCAs previously were reported to exhibit a significant antityrosinase activity in vitro (J. Y. Lim et al., Phytother. Res., 13, 371-5 (1999); H. S. Lee, J. Agric. Food Chem., 50, 1400-03 (2002)).

Studies demonstrated the superiority of HCAs in inhibiting tyrosinase compared to other commercially available tyrosinase inhibitors, and especially over kojic acid (N. Dayan et al., AAPS Annual Meeting, Abstract, USA, AM-04-0053 (2004)). Hydroxycinnamic acid also has other advantages that make the compound an excellent candidate for use as a skin-lightening agent. These advantages include an abundant supply because it is plant derived, of high purity, nonirritating, antimutagenic with protective properties, stable (no degradation after six month's storage at 50° C.), does not color degrade, and is bacteriostatic with preservative properties.

Although HCAs are considered safe and effective compounds for inhibiting tyrosinase and blocking melanogenesis, HCAs are not used in cosmetic or dermatologic formulations to treat hyperpigmentation. The lack of commercialization is attributed to the physical properties of HCAs, particularly the difficulties in solubilizing an HCA in a carrier, which can adversely affect bioavailability, i.e., an insufficient skin penetration to reach the melanocytes.

The present invention is directed to providing compositions that overcome problems associated with prior tyrosinase inhibitors used to treat hyperpigmentation. The compositions contain an HCA, and overcome problems associated with incorporating an HCA into a consumer acceptable skin-lightening composition.

### SUMMARY OF THE INVENTION

The present invention is directed to cosmetic and dermatological compositions used in a method of treating hyperpigmentation. More particularly, the present invention is directed to compositions that demonstrate an enhanced ability to lighten skin color because of an improved permeability of the active agent through the surface of the skin.

In accordance with the present invention, the active agent, i.e., an HCA, is dissolved in an organic compound having one or more hydroxy groups, a silicone fluid, or a mixture thereof. The compound having a hydroxy group can be an organic solvent or a surfactant, e.g., a monoC₁₋₄alkyl ether of an ethylene glycol oligomer or a monoC₁₋₄alkyl ether of a propylene glycol oligomer. The resulting solution typically is incorporated into a cosmetic or a dermatological composition for topical application to the skin.

Therefore, one aspect of the present invention is to provide a composition comprising about 0.01% to about 30%, by weight, of an HCA dissolved in an organic compound having one or more hydroxy groups, a silicone fluid, or a mixture thereof. The resulting solution can be applied directly to the skin, or can be incorporated into a cosmetic or dermatological formulation, for example, an oil-in-water emulsion, a water-in-oil emulsion, or a gel.

Another aspect of the present method is to provide a method of treating hyperpigmentation in a mammal, including humans, comprising applying a composition comprising an HCA dissolved in an organic compound having one or more hydroxy groups, a silicone fluid, or a mixture thereof, to a skin surface of the mammal. The method is capable of lightening dark skin attributed to age spots or a melasma, for example.

In accordance with the present invention, the HCA is admixed with an organic compound having one or more hydroxy groups, a silicone fluid, or a mixture thereof, in particular, a monoC₁₋₄alkyl ether of an ethylene glycol oligomer or a monoC₁₋₄alkyl ether of a propylene glycol oligomer, to provide a solution suitable for application to the skin of a mammal, including the scalp. This solution can be incorporated into a cosmetic formulation, or cosmetic formulation ingredients can be added thereto, for an efficient and efficacious application of the HCA to the skin.

Yet another aspect of the present invention is to provide a composition containing an HCA dissolved in an organic compound having one or more hydroxy groups and/or a silicone fluid, and use of the composition as a skin care product, a topical drug product, or a cosmetic product.

These and other aspects and novel features of the present invention will become apparent from the following detailed description of the preferred embodiments.

### BRIEF DESCRIPTION OF THE FIGURES

FIG. 1 contains plots of amounts of p-hydroxycinnamic acid (p-HCA) permeating the skin (in μg/cm²) vs. time for a composition of the present invention and for a control composition;

FIG. 2 contains bar graphs comparing the ability of p-HCA and kojic acid, at varying concentrations, to inhibit tyrosinase; and

FIG. 3 and FIG. 4 contain plots of chromameter readings vs. time comparing the ability of p-HCA in ethoxydiglycol to kojic acid in a clinical study directed to skin brightening efficacy (FIG. 3) and a reduction in skin redness (FIG. 4).

### DETAILED DESCRIPTION OF THE PREFERRED EMBODIMENTS

An agent for treating hyperpigmentation often acts by inhibiting the biosynthesis of melanins. One example is to inhibit tyrosinase activity, and thereby preclude conversion of tyrosine to melanin. A number of tyrosinase inhibitors are known, and some have been used to treat hyperpigmentation, i.e., to lighten skin.

Among the most potent tyrosinase inhibitors are the HCAs, which also are nontoxic and nonirritating. Some HCAs, like p-hydroxycinnamic acid (p-HCA), can be found in fruits and vegetables, and presently are being used in the food industry as antioxidants.

p-Hydroxycinnamic acid is a phenolic cinnamic acid derivative that inhibits the development of cancer, and is found in various plants such as tomatoes, green peppers, carrots, strawberries, and pineapples, as well as herbal plants, like basil and turmeric. p-Hydroxycinnamic acid is activated during digestion and interferes with the development of cancer-causing nitrosamines. p-Hydroxycinnamic acid also is used in the cosmetic industry as a bacteriostat.

Irrespective of these positive attributes, no commercial skin lightening composition including an HCA in general, or p-HCA in particular, has been introduced. The reasons for this lack of a commercial product containing an HCA are two-fold. First, HCAs have poor skin permeability. Second, HCAs are highly insoluble in solvents and carriers typically used in skin care and dermatological compositions. The present invention overcomes these deficiencies and permits the use of an HCA in treating hyperpigmentation.

As used herein, the term “hyperpigmentation” is an actual or a perceived skin impairment of excessive dark color. The skin impairment can be actual, i.e., attributed to age, excessive sun exposure, or a disease or condition leading to dark skin areas. The dark skin areas can be in the form of spots, blotches, or relatively large areas of dark color. The skin impairment also can be perceived, i.e., a perception by an individual that his/her skin shade is too dark and the individual has a cosmetic desire to lighten the skin shade.

Accordingly, compositions of the present invention are useful in treating a variety of skin hyperpigmentations, for example, depigmenting melasma, i.e., dark patches of pigmentation on the face and other parts of the body, or for voluntary whitening skin pigmentation.

Typically, the dark skin impairment is attributed to an elevated level of melanin. In accordance with the present invention, the composition and method can be used to treat hyperpigmentation, i.e., to lighten dark skin, or to prevent hyperpigmentation, i.e., to reduce or eliminate the production of excessive amounts of melanin and thereby preclude darkening of skin. Therefore, the present invention is directed to a composition comprising an HCA dissolved in a hydroxy-containing compound, e.g., a monoC₁₋₄alkyl ether of an ethylene glycol oligomer or a monoC₁₋₄alkyl ether of a propylene glycol oligomer, a silicone fluid, or a mixture thereof, and use of the composition in a method of treating hyperpigmentation.

The active agent in a present composition and method is an HCA. In accordance with the present invention, an HCA is dissolved in an organic compound having one or more hydroxy groups, a silicone fluid, or a mixture thereof. The organic compound containing one or more hydroxy groups can be a surfactant or an organic solvent. The compound containing one or more hydroxy group can contain one to six hydroxy groups, and typically contains one to three hydroxy groups.

In one embodiment, the HCA is dissolved in a monoC₁₋₄alkyl ether of an ethylene glycol oligomer or a monoC₁₋₄alkyl ether of a propylene glycol oligomer, also collectively termed herein as a “monoC₁₋₄-alkyl ether.” In other embodiments, the HCA is dissolved in a C₂₋₄ alcohol, a C₃₋₅ glycol, a polyethylene glycol, a polypropylene glycol, a triol, a polyol, an ethoxylated glycerin, or mixtures thereof. Nonlimiting examples of useful compounds include propylene glycol, butylene glycol, pentylene glycol, glycereth-7 through glycereth-31, PEG-4 through PEG-800, PPG-3 through PPG-69, ethanol, isopropyl alcohol, n-propanol, n-butanol, sec-butanol, t-butyl alcohol, diethylene glycol, dipropylene glycol, tripropylene glycol, hexylene glycol, 1,2,6-hexanetriol, sorbitol, and mixtures thereof.

Useful surfactants having one or more hydroxy groups include nonionic surfactants, not limited to, ethoxylated octyl phenols, ethoxylated nonyl phenols, ethoxylated linear C₈₋₂₂ alcohols, propoxylated linear C₈₋₂₂ alcohols, ethoxylated and propoxylated C₈₋₂₂ alcohols, polyethylene glycol ethers of sorbitol, ethylene oxide-propylene oxide block copolymers, or mixtures thereof.

Useful silicone fluids include linear and cyclic, volatile and nonvolatile, dimethyl siloxane fluids, including siloxane fluids having phenyl substituents. Useful silicone fluids are disclosed in U.S. Pat. No. 5,456,863, incorporated herein by reference. Exemplary siloxanes include phenyltrimethicone, cyclic or linear, low molecular weight, volatile polydimethylsiloxanes known as cyclomethicones and dimethicones, respectively, and methicones. The cyclomethicones are low viscosity, low molecular weight, water-insoluble cyclic compounds having an average of about 3 to about 6-[O—Si(CH₃)₂]-repeating group units per molecule. Cyclomethicones are available commercially under the tradenames SILICONE 344 FLUID and SILICONE 345 FLUID from Dow Corning Corporation, Midland, Mich. and SILICONE SF-1173 and SILICONE SF-1202 from General Electric, Waterford, N.Y., for example.

An example of a linear, low-molecular weight, volatile dimethicone is the compound hexamethyldisiloxane, available commercially under the tradename DOW CORNING 200 FLUID, from Dow Corning Corp., Midland, Mich. DOW CORNING 200 FLUID has a viscosity of 0.65 cs (centistokes). Other linear polydimethylsiloxanes, such as decamethyltetrasiloxane, octamethyltrisiloxane, and dodecamethylpentasiloxane, also are useful. Other useful linear siloxanes are hexyl dimethicone, polyphenylmethyl-siloxane, and bisphenylhexamethicone. Nonvolatile siloxanes also can be used.

The resulting solution contains about 0.01% to about 30%, by weight, of an HCA. The composition can be used as is, diluted, or admixed with other composition ingredients known in the cosmetic and dermatologic arts to provide an efficacious and esthetic composition for topical application to the skin.

The HCA can be, but is not limited to, 2-, 3-, or 4-hydroxycinnamic acids; 2,3-, 2,4-, or 3-4-dihydroxycinnamic acid; 2-, 3-, or 4-methoxycinnamic acid; 3-hydroxy-4-methoxycinnamic acid; 4-hydroxy-3-methoxycinnamic acid; or mixtures thereof. Preferably, the HCA comprises 2-, 3-, or 4-hydroxycinnamic acid. To achieve the full advantage of the present invention, the HCA comprises 4-hydroxycinnamic acid.

For the sake of brevity, the following disclosure is particularly directed to p-hydroxycinnamic acid, also known as p-coumaric acid, and referred to herein as “p-HCA.” It should be understood that other HCAs can be substituted for p-HCA in a composition or method of the present invention.

The above-described HCA solution can be applied directly to skin. In this case, the amount of HCA in the solution typically is about 0.01% to about 10%, and preferably about 0.05% to about 5%, by weight of the solution. The above-described HCA solution also can be diluted with a solvent or other carrier prior to application to the skin. The diluting solvent can be the same or different from the organic compound having one or more hydroxy group or a silicone fluid. The diluting solvent should not cause precipitation of the HCA from the final solution, or otherwise adversely affect the ability of the HCA in solution to penetrate the surface of the skin and treat hyperpigmentation.

The above-described HCA solution also can be formulated into various product forms, such as dermal patches, emulsions, or gels, by the addition of formulation ingredients to the HCA solution, or addition of the HCA solution to the formulation ingredients. Nonlimiting formulation ingredients and product forms are discussed below, and do not adversely affect the ability of the HCA solution to treat hyperpigmentation.

An HCA is dissolved in an organic compound having one or more hydroxy groups and/or a silicone fluid, like a monoC₁₋₄alkyl ether of an ethylene glycol oligomer or a monoC₁₋₄alkyl ether of a propylene glycol oligomer. As discussed below, dissolving the HCA in a suitable hydroxy-containing compound improves penetration of the HCA through the skin surface and permits a degree of skin lightening that previously could not be attained using an HCA.

A monoC₁₋₄alkyl ether of an ethylene glycol oligomer or a monoC₁₋₄alkyl ether of a propylene glycol oligomer used to dissolve HCA has a general formula:

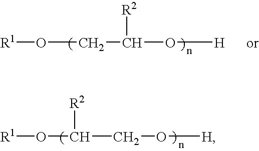

wherein R¹ is C₁₋₄alkyl, R² is hydrogen or methyl, and n is 2 or 3. For example, R¹ is methyl, ethyl, isopropyl, n-propyl, n-butyl, sec-butyl, isobutyl, or terbutyl.

Nonlimiting examples of a monoC₁₋₄alkyl ether of an ethylene glycol or a monoC₁₋₄alkyl ether of a propylene glycol oligomer include, but are not limited to, ethoxydiglycol, methoxydiglycol, butoxy-diglycol, methoxytriglycol, ethoxytriglycol, and mixtures thereof. Preferred monoC₁₋₄alkyl ethers include ethoxydiglycol and methoxydiglycol. To achieve the full advantage of the present invention, the monoC₁₋₄alkyl ether comprises ethoxydiglycol.

As previously stated, a solution containing the HCA and the organic compound having one or more hydroxy groups, a silicone fluid, or mixture thereof, can be applied as is, after dilution, or after incorporation into a cosmetic or dermatological formulation. The final composition also can contain an optional second active skin-lightening agent. Useful second active skin-lightening agents include, but are not limited to, skin exfoliants; kojic acid; retinoic acid; hydroquinone or a derivative thereof, such as benzylhydroquinone ether; ascorbic acid or a derivative thereof, such as magnesium ascorbyl phosphate; a caffeic acid or ester thereof; a benzofuran, such as 5- or 6-hydroxybenzofuran; a plant extract, such as licorice, mulberry, heather, and angelica ashitaba; a pearl extract; a steroidal antiinflammatory agent of the hydrocortisone-type and the like; a nonsteroidal antiinflammatory agent selected from the group consisting of acetylsalicylic acid, acetaminophen, naproxen, and fenamic acid derivatives, such as the sodium salt; an antiinflammatory agent, such as alpha-bisabolol, beta-glycyrrhetinic acid, allantoin, aloe extract, rosmarinic acid, azulene or a derivative thereof, asiaticoside, sericoside, ruscogenin, escin, escolin, quercetin, rutin, betulinic acid or a derivative thereof, catechin or a derivative thereof; and mixtures thereof.

The HCA solution containing a hydroxy-containing compound is useful in personal care, cosmetic, and pharmaceutical compositions. The present solutions provide an effective delivery of an HCA to lighten the skin. The resulting compositions for skin lightening can be formulated with other topically applied active compounds, in addition to or in lieu of an optional second active skin-lightening agent to achieve both skin lightening and a second cosmetic or therapeutic effect different from skin lightening.

In accordance with an important feature of the present invention, a topically applied compound for providing a second cosmetic or therapeutic effect can be any of a wide variety of compounds, either water soluble or oil soluble.

Such a topically applied active compound, therefore, can be one of, or a mixture of, a cosmetic compound, a medicinally active compound, a compound used in cosmetics or personal care, or any other compound that is useful upon topical application to the skin. Such topically active agents include, but are not limited to, skin-care compounds, plant extracts, antioxidants, insect repellants, counterirritants, vitamins, steroids, antibacterial compounds, antifungal compounds, antiinflammatory compounds, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to:

myristyl propionate,

isopropyl isostearate,

isopropyl myristate,

isopropyl palmitate,

cetyl acetate,

cetyl propionate,

cetyl stearate,

isodecyl neopentanoate,

cetyl octanoate,

isocetyl stearate;

(b) aliphatic di- and tri-esters of polycarboxylic acid, including, but not limited to:

diisopropyl adipate,

diisostearyl fumarate,

dioctyl adipate, and

triisostearyl citrate;

(c) aliphatic polyhydric alcohol esters, including, but not limited to:

propylene glycol dipelargonate;

(d) aliphatic esters of aromatic acids, including, but not limited to:

C₁₂-C₁₅ alcohol esters of benzoic acid,

octyl salicylate,

sucrose benzoate, and

dioctyl phthalate.

Numerous other esters are listed in the CTFA Handbook, at pages 24 through 26, incorporated herein by reference.

The topically applied compound also can be an antioxidant or an optical brightener, like a distyrylbiphenyl derivative, stilbene or a stilbene derivative, a pyralozine derivative, or a coumarin derivative. Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine.

The optical brighteners are available under a variety of tradenames, such as TINOPAL®, LCUCOPHOR®, and CALCOFLUOR®. Specific fluorescent compounds include, but are not limited to, TINOPAL® 5BM, CALCOFLUOR® CG, and LEUCOPHOR® SB.

In addition, other compounds can be included in a present composition as the topically active compound in an amount sufficient to perform their intended function. For example, sunscreen compounds such as benzophenone-3, tannic acid, uric acids, quinine salts, dihydroxy naphtholic acid, an anthranilate, p-aminobenzoic acid, phenylbenzimidazole sulfonic acid, PEG-25, or p-aminobenzoic acid can be used as the topically applied compound. Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

Similarly, topically applied drugs, like antifungal compounds, antibacterial compounds, anti-inflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polymyxin b sulfate-bacitracin, zinc-neomycin sulfate-hydrocortisone, chloramphenicol, ethyl-benzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The topically active compound also can be a plant extract or a natural oil. Numerous plant extracts are available from Lipo Chemicals, Inc. Paterson, N.J. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

A composition of the present invention is prepared by dissolving the HCA in an organic compound having one or more hydroxy groups, a silicone fluid, or a mixture thereof, like a monoC₁₋₄alkyl ether. The present compositions can be admixed with other ingredients traditionally included in cosmetic, dermatological, medicinal, and other such compositions. These ingredients include, but are not limited to, dyes, fragrances, preservatives, antioxidants, detackifying agents, and similar types of compounds. The ingredients are included in the composition in an amount sufficient to perform their intended function.

The following additional ingredients typically are included in a present composition, in combination with a solution of an HCA in a monoC₁₋₄-alkyl ether. Each of these ingredients, and any other ingredient, is present in a sufficient amount to perform its intended function, without adversely affecting the efficacy of HCA with respect to treating hyperpigmentation.

For example, a present composition can contain a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

A present composition also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A present composition also can contain an additional organic solvent. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4,1,5-pentanediol, similar hydroxyl-containing compounds, and mixtures thereof. The solvent also can be water or an aprotic solvent, e.g., dimethyl sulfoxide or tetrahydrofuran.

A present composition also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Other classes of optional ingredients included in a present composition can be, but not limited to, pH adjusters, chelating agents, preservatives, buffering agents, foam stabilizers, opacifiers, and similar classes of ingredients known to persons skilled in the art. Specific optional ingredients include inorganic phosphates, sulfates, and carbonates as buffering agents, EDTA and phosphates as chelating agents; and acids and bases as pH adjusters.

Nonlimiting examples of basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters are the mineral acids and organic carboxylic acids. Nonlimiting examples of mineral acids are citric acid, hydrochloric acid, nitric acid, phosphoric acid, and sulfuric acid.

In the personal care area, a solution of an HCA in an organic compound having one or more hydroxy groups, a silicone fluid, or a mixture thereof, can be incorporated into compositions designed as cosmetic basecoats and undercoats, bath capsules, bath oils, bath tablets, bath salts, bath soaps, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin lighteners, tonics, dressings, and wrinkle smoothing creams and lotions.

In particular, a present solution of an HCA in a hydroxy-containing compound can be incorporated into lotions; makeup preparations, like makeup foundations; skin care preparations, like hand lotions, vanishing creams, night creams, sunscreens, body lotions, facial creams, clay masks, moisturizing lotions, make-up removers, antiacne preparations, antiaging preparations, and sebum control; analgesic and cortisonal steroid creams and preparations; insect repellants; and facial masks and revitalizers. The compositions also can be incorporated into plasters, bandages, dressings, gauze pads, and similar articles.

The final composition can be in the form of a solution, oil-in-water emulsion, water-in-oil emulsion, gel, or other product form known in the skin care and dermatological arts. The composition form can be, for example, a liquid form, e.g., a solution, a gelled solution, or a suspension in an aqueous or oily medium; or a semi-liquid formulation, e.g., a cream, a gel, a paste, an ointment, a salve, a liposome, an emulsion, or a microemulsion.

A composition of the present invention is topically applied to the skin as needed in order to lighten skin color to a desired degree. Typically, the composition is topically applied to the skin one to four times per day. However, application of a present composition can be more or less frequent as prescribed, required, or desired. The present compositions are applied to the skin by spraying or rubbing. The preferred route of administration is rubbing onto the skin with a soft massage to ensure intimate contact with the skin.

To demonstrate the new and unexpected benefits provided by a composition and method of the present invention, the following tests were performed. First, to confirm in vivo activity of p-HCA, the metabolism of p-HCA in the skin was determined. In this test, human skin was homogenized in a PBS (phosphate-buffered saline)-ethanol solution, followed by dissolution of p-HCA. The solution was incubated at 37° C. for 8 hours. Samples were taken after 0, 4, and 8 hours, and were centrifuged. The supernatant was filtered and analyzed by HPLC. It was found that after eight hours of incubation with skin enzymes, that only 3.6% of the p-HCA was metabolized. The amount of p-HCA that is metabolized was considered insignificant with respect to an efficacious treatment of hyperpigmentation.

In order to inhibit tyrosinase to prevent the formation of melanin, the HCA must penetrate the skin a sufficient degree to contact the enzyme at the site of action, i.e., melanosomes located in the melanocytes. To effectively penetrate the skin to the target site, the HCA must be solubilized. HCAs are insoluble compounds, and it has been found that solubilizing an HCA in a monoC₁₋₄alkyl ether, then incorporating the resulting solution into a formulation for topical application, or using the resulting composition as is or after dilution, enhances penetration of the HCA to the melanosomes.

Various solvents were tested for an ability to dissolve p-HCA and provide a suitable composition for topical application to efficaciously treat hyperpigmentation. p-HCA was added to various solvents, then sonicated for two hours at 35° C. until no further HCA was dissolved. The samples were cooled to room temperature (25° C.) and allowed to stabilize for 15 hours. The undissolved p-HCA was allowed to settle, the supernatant liquid was filtered, and diluted with the HPLC mobile phase for assay. All samples were analyzed by HPLC on a Hypersil ODS C18 column using water, methanol, glacial acetic acid (55:45:1) as the mobile phase. The flow rate was 1 ml/min and the wavelength for detection was 270 nm. In this test, it was found that p-HCA had a solubility in ethoxydiglycol at 25° C. of 19.7%, by weight.

In another experiment, a 10% solution of p-HCA in ethoxydiglycol first was prepared. This solution was added to a skin care emulsion in a sufficient amount to provide either 0.3% or 0.1%, by weight, of p-HCA in the final composition. As a comparative example, 0.3%, by weight, of solid p-HCA was added to the same skin care emulsion. The skin care emulsion to which solid p-HCA or the p-HCA solution was added had the following formula (w/w %):
85.35 water
0.2 methylparaben
0.35 xanthan gum
5.0 butylene glycol
4.0 sesame oil
2.5 glyceryl stearate

0.5 PEG-150 distearate
1.25 neopentyl glycol dicaprylate/dicaprate
0.6 emulsifying wax
0.25 imidazolidinyl urea.

The skin care emulsions containing p-HCA were applied to cadaver skin. Skin penetration of p-HCA was studied using Franz diffusion cells, n=5 (Permegear, Bethlehem, Pa.). The receptor contained an isotonic phosphate buffer with 30% ethanol, and was stirred continuously at 600 rpm. Samples of dermatomed human cadaver skin (NDRA, Philadelphia) were placed on the cells and prehydrated for 1 hour prior to the experiment. One milliliter of each formulation was added to the donor compartment of each cell, which was covered tightly with PARAFILM®. Samples (300 μl) were withdrawn from the receptor compartment every hour over 8 hours, and replaced with 300 μl of fresh receptor solution. After 8 hours, the skin was removed and cut into small pieces. The skin pieces were homogenized using a Kinematica POLYTRON® homogenizer and centrifuged. The supernatant was filtered through 0.22μ filters, and the p-HCA content was quantified.

All samples were analyzed by HPLC on a Hypersil ODS C18 column using water, methanol, glacial acetic acid (55:45:1) as the mobile phase at a flow rate of 1 ml/min and detection at 270 nm. Lag time and flux were obtained from permeation profiles and skin drug content also was recorded.

TABLE 1

| | | Amount | |
|---|---|---|---|
| | % p-HCA | p-HCA | |
| | permeated | remaining | |
| | through | within the skin | Flux |
| | the | (μg/g of | (μg/cm² × |
| | skin | skin) | hr.) |
| 0.3% p-HCA incorporated in | 0.85 | 1097 | 5.43 |
| powder form (comparative) | | | |
| 0.3% p-HCA incorporated as a | 1.50 | 2110 | 11.25 |
| 10% solution in ethoxydiglycol | | | |
| 0.1% p-HCA incorporated as a | 0.50 | 1148 | 1.42 |
| 10% solution in ethoxydiglycol | | | |

Table 1 summarizes the ethoxydiglycol enhanced permeation of p-HCA through the skin. In presence of ethoxydiglycol, the percent of p-RCA that permeates cadaver skin was elevated by 43%, and the percentage of p-HCA that remained within the skin was elevated by about 50%. The flux of p-HCA through the skin also was doubled.

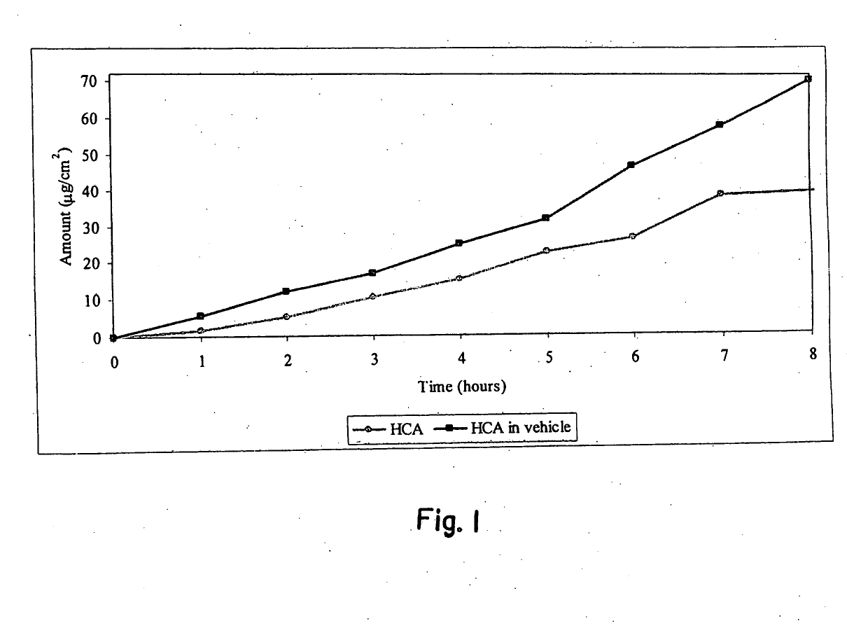

FIG. 1 further illustrates the results of Table 1 in graph form. FIG. 1 shows that the amount of p-HCA permeating through the skin to the lower epidermis, in μg/cm², where melanocytes reside, in this in vitro test was substantially greater when the p-HCA first is solubilized in a monoC₁₋₄alkyl ether, as opposed to adding the p-HCA to the emulsified composition as a solid. FIG. 1 and Table 1 show that ethoxydiglycol increased the flux into the skin by 107%, and the deposition of p-H(CA in the skin was elevated by 93%.

Additional hydroxy-containing compounds were tested for an ability to solubilize an HCA and allow the HCA to permeate the surface of skin and deposit the HCA at the site of melanin formation. In this test, solubility of HCA in the compounds determined as follows.

p-Hydroxycinnamic acid was added to the hydroxy-containing compound and sonicated until no further p-HCA dissolved after two hours of sonication at 35° C. The test sample was cooled to room temperature, then stabilized for 15 hours. After the p-HCA was allowed to settle, the supernatant was filtered through a 0.2 micron syringe filter. Approximately 0.08 g of this filtered material was weighed into a 25 ml volumetric flask and diluted to volume with HPLC mobile phase. Two standards were analyzed by HPLC and compared to the two samples. The HPLC assay was identical to the HPLC assay discussed above.

It was found that p-HCA had the following solubilities, in wt %:
Hydroethanolic solutions/gels: e.g., 30% hydroethanolic solution: 0.75%;
Ethanol: 14.8%;
PEG-8: 5.4%;
pentylene glycol: 1.75%;
butylene glycol: 2.25%;
glycereth-26: 7%;
silicone fluid DC 193: 4.7.

The following formula was tested for skin penetration:
Water 28.1% (by wt);
Carbopol 981 (2%) 17.0%;
Ethanol SDA 40b-190 53.0%;
Glycerin 1.0%;
Triethanolamine 0.4%;
p-Hydroxycinnamic acid 0.5%.

The method used to determine skin permeability was the Franz diffusion method disclosed above. The test results showed that the above formula deposited an amount of HCA in the skin that is four times greater than the amount transmitted through the skin, which demonstrates a targeted delivery of p-HCA to the site of melanin formation. In particular, 3.9 mcg/ml of p-HCA was found in the receptor compartment of the skin (i.e., permeated through the skin) as opposed to 16.3 mcg/ml p-HCA found within the skin.

In one embodiment of the invention, a 15% (by wt) solution of p-HCA in ethoxydiglycol was prepared. Typically, such a solution is incorporated into a personal care, cosmetic, or dermatologic composition in an amount of about 1% to about 10%, and preferably about 2% to about 6%, by weight, to provide a final composition-containing about 0.15% to about 0.15%, and preferably 0.3% to about 0.9%, by weight, p-HCA.

In another test, the ability of a solution of p-HCA in ethoxydiglycol to inhibit tyrosinase in vitro was determined. In this test, a tyrosinase solution and either a test composition or a control were mixed in a spectrophotometer cell, and the cell was placed in a UV-visible spectrophotometer. Tyrosine was added as a substrate and the absorbance was recorded for 30 minutes. Percent inhibition was calculated from the slope of the inhibition curve and was normalized to the control.

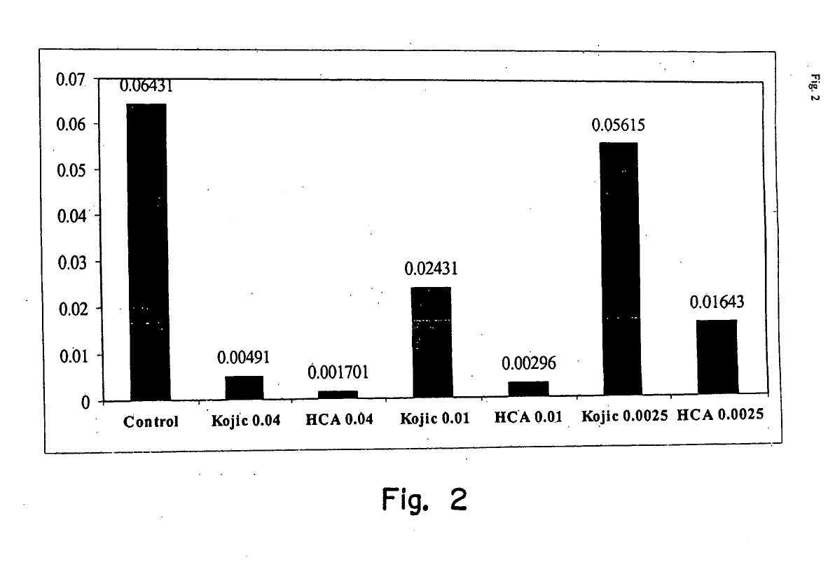

FIG. 2 shows that p-HCA in ethoxydiglycol is superior to kojic acid with respect to inhibiting tyrosinase. The bar graphs of FIG. 2 specifically show that p-HCA in ethoxydiglycol inhibits tyrosinase about four to five times greater than kojic acid.

The skin brightening efficacy of p-HCA in ethoxydyglycol versus kojic acid also was tested in a clinical study. In this study, an emulsion containing 0.3%, by weight, p-HCA or kojic acid was used on twenty, mostly female, subjects between the ages of 30 to 50 years. The test composition was applied to the abdomen twice daily (a.m. and p.m.). The test was conducted for eight weeks, and chromameter readings were taken at baseline, and after 2, 4, 6, and 8 weeks.

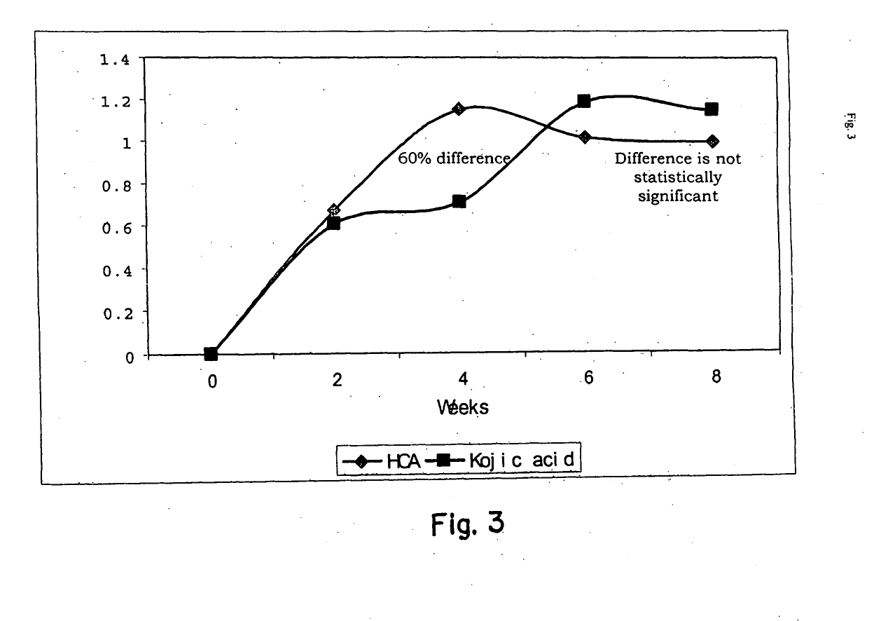

FIG. 3 illustrates that p-HCA in ethoxydiglycol is more effective than kojic acid over weeks 2 through 5 of the study (i.e., a faster skin-lightening effect, including a 60% improvement at week 4), and matches the effectiveness of kojic acid from week 6-through week 8 of the study.

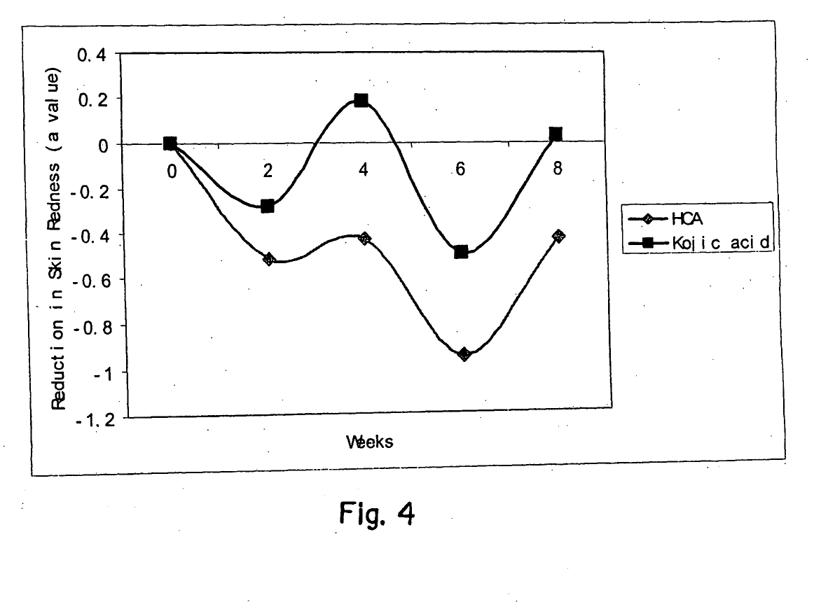

FIG. 4 illustrates the results of an additional test showing the reduction in skin redness observed in the clinical trial. p-HCA in ethoxydiglycol demonstrated a reduced redness throughout the eight-week clinical-trial. In contrast, kojic acid did not reduce redness, but actually induced redness at weeks 4 and 8.

Obviously, many modifications and variations of the invention as hereinbefore set forth can be made without departing from the spirit and scope thereof and, therefore, only such limitations should be imposed as are indicated by the appended claims.

1. A composition comprising about 0.01% to about 30%, by weight, of a hydroxycinnamic acid, a methoxycinnamic acid, or mixtures thereof dissolved in a compound having one or more hydroxy groups.

2. The composition of claim 1 comprising about 0.1% to about 20% of the hydroxycinnamic acid, methoxycinnamic acid, or mixtures thereof.

3. The composition of claim 1 comprising about 0.5% to about 15% of the hydroxycinnamic acid, methoxycinnamic acid, or mixtures thereof.

4. The composition of claim 1 wherein the hydroxycinnamic acid or methoxycinnamic acid is selected from the group consisting of 2-hydroxycinnamic acid, 3-hydroxycinnamic acid, 4-hydroxycinnamic acid, 2,3-dihydroxycinnamic acid, 2,4-dihydroxycinnamic acid, 3,4-dihydroxycinnamic acid, 2-methoxycinnamic acid, 3-methoxycinnamic acid, 4-methoxycinnamic acid, 3-hydroxy-4-methoxycinnamic acid, 3-methoxy-4-hydroxycinnamic acid, and mixtures thereof.

5. The composition of claim 1 wherein the hydroxycinnamic acid is selected from the group consisting of 2-hydroxycinnamic acid, 3-hydroxycinnamic acid, 4-hydroxycinnamic acid, and mixtures thereof.

6. The composition of claim 1 wherein the hydroxycinnamic acid comprises 4-hydroxycinnamic acid.

7. The composition of claim 1 wherein the compound having one or more hydroxy groups comprises a monoC₁₋₄alkyl ether of an ethylene glycol oligomer, a monoC₁₋₄alkyl ether of a propylene glycol oligomer, or mixtures thereof.

8. The composition of claim 7 wherein the monoC₁₋₄alkyl ether of an ethylene glycol oligomer or a monoC₁₋₄alkyl ether of a propylene glycol oligomer has a general formula 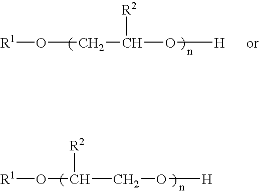
wherein R¹ is C₁₋₄alkyl, R² is hydrogen or methyl, or n is 2 or 3.

9. The composition of claim 7 wherein the monoC₁₋₄alkyl ether of an ethylene glycol oligomer or a monoC₁₋₄alkyl ether of a propylene glycol oligomer is selected from the group consisting of ethoxydiglycol, methoxydiglycol, butoxydiglycol, methoxytriglycol, ethoxytriglycol, and mixtures thereof.

10. The composition of claim 7 wherein the monoC₁₋₄alkyl ether of an ethylene glycol oligomer or a monoC₁₋₄alkyl ether of a propylene glycol oligomer comprises ethoxydiglycol.

11. The composition of claim 1 wherein the compound having one or more hydroxy groups is selected from the group consisting of a C₂₋₄ alcohol, a C₃₋₅ glycol, a polyethylene glycol, a polypropylene glycol, a triol, a polyol, an ethoxylated glycerin, and mixtures thereof.

12. The composition of claim 11 wherein the compound having one or more hydroxy groups is selected from the group consisting of propylene glycol, butylene glycol, pentylene glycol, glycereth-7 through glycereth-31, PEG-4 through PEG-800, PPG-3 through PPG-69, ethanol, isopropyl alcohol, n-propanol, n-butanol, sec-butanol, t-butyl alcohol, diethylene glycol, dipropylene glycol, tripropylene glycol, hexylene glycol, 1,2,6-hexanetriol, sorbitol, and mixtures thereof.

13. The composition of claim 1 wherein the compound having one or more hydroxy groups comprises a nonionic surfactant.

14. The composition of claim 13 wherein the nonionic surfactant is selected from the group consisting of an ethoxylated octyl phenol, an ethoxylated nonyl phenol, an ethoxylated linear C₈₋₂₂ alcohol, a propoxylated linear C₈₋₂₂ alcohol, an ethoxylated and propoxylated linear C₈₋₂₂ alcohol, a polyethylene glycol ether of sorbitol, an ethylene oxide-propylene oxide block copolymer, and mixtures thereof.

15. (canceled)

16. A skin care formulation comprising a composition of claim 1.

17. The skin care formulation of claim 16 in a form of a solution, an emulsion, or a gel.

18. The skin care formulation of claim 16 further comprising a topically applied compound selected from the group consisting of a pesticide, a drug, a therapeutic agent, a deodorant, a skin conditioner, an antioxidant, an insect repellant, a counterirritant, a vitamin, a plant extract, a steroid, an antibacterial compound, an antifungal compound, an antiinflammatory compound, a topical anesthetic, an epidermal lipid replacement, a sunscreen, an optical brightener, a dermatitis or skin disease medication, and mixtures thereof.

19. The skin care formulation of claim 18 wherein the topically applied compound is selected from the group consisting of benzocaine, dyclonine hydrochloride, aloe vera, butamben picrate, lidocaine hydrochloride, xylocaine, providone-iodine, polymyxin b sulfate-bacitracin, zinc-neomycin sulfate-hydrocortisone, chloramphenicol, ethylbenzethonium chloride, erythromycin, lindane, benzoyl peroxide, erythromycin benzoyl peroxide, clindamycin phosphate, 5,7-dichloro-8-hydroxyquinoline, alclometasone dipropionate, betamethasone valerate, o-amino-p-toluenesulfonamide monoacetate, monobenzone, amcinonide, diflorasone diacetate, hydrocortisone, methylbenzethonium chloride, PEG-4 dilaurate, lanolin oil, petrolatum, mineral wax, butocouazole nitrate, haloprogin, clotrimazole, O-[(2-hydroxymethyl)methyl]guanine, alclometasone dipropionate, betamethasone valerate, isopropyl myristate MSD, anthralin, methoxsalen, coal tar, 2-(acetyloxy)-9-fluoro-1′,2′,3′,4′-tetrahydro-1-hydroxypregna-1,4-dieno-[16,17-b]naphthalene-3,20-dione, 21-chloro-9-fluoro-1′,2′,3′,4′-tetrahydro-11 b-hydroxypregna-1,4-dieno-[16z, 17-b]naphthalene-3,20-dione, allantoin, salicylic acid, isopropyl myristate, ascorbic acid, retinol, salicylic acid, zinc pyrithione, benzophenone-3, a fragrance, glycolic acid, hyalauronic acid, hydrogen peroxide, a protein, an enzyme, tocopherol, butein, hydroquinone, kojic acid, jojoba oil, an alpha or beta hydroxy acid, and mixtures thereof.

20. The skin care formulation of claim 16 further comprising a second active skin lightening compound selected from the group consisting of a skin exfoliant, kojic acid, retinoic acid, hydroquinone or a derivative thereof, ascorbic acid or a derivative thereof, a caffeic acid or an ester thereof, a benzofuran, a plant extract, a pearl extract, a hydrocortisone-type steroidal antiinflammatory agent, a nonsteroidal antiinflammatory agent selected from the group consisting of acetylsalicylic acid, acetaminophen, naproxen, and fenamic acid derivatives, an antiinflammatory agent selected from the group consisting of alpha-bisabolol, beta-glycyrrhetinic acid, allantoin, aloe extract, rosmarinic acid, azulene or a derivative thereof, asiaticoside, sericoside, ruscogenin, escin, escolin, quercetin, rutin, and betulinic acid or a derivative thereof, and mixtures thereof.

21. A method of treating human skin comprising a step of contacting the skin with a composition of claim 1 in a sufficient amount to lighten skin color.

22. (canceled)

23. The method of claim 21 wherein the hydroxycinnamic acid or methoxycinnamic acid penetrates a surface of the skin to an epidermis or dermis.

24. The method of claim 21 wherein the amount of hydroxycinnamic acid or methoxycinnamic acid penetrating the skin to the melanocytes is about 40% greater compared to applying a composition to the skin wherein the hydroxycinnamic acid or methoxycinnamic acid is added to the composition as a solid.

25. The method of claim 21 wherein the human skin is hyperpigmented.

26. The method of claim 25 wherein the hyperpigmentation is attributed to drug use, cyanic melasma, senile melasma, vitiglio, adverse sequelae following sclerotherapy, a postinflammatory response, a posttraumatic response, pregnancy, estro-progestative contraception, excessive sun exposure, photosensitization, postlesional cicatrization, Addison's disease, or an intrinsic genetic profile.

27. A method of treating hyperpigmented mammalian skin comprising a step of contacting the skin with a formulation comprising a composition of claim 1 in a sufficient amount to lighten skin color.

28. A method of preventing hyperpigmented mammalian skin comprising a step of contacting the skin with a composition of claim 1 in a sufficient amount to prevent excessive darkening of the skin.

29. A method of preventing hyperpigmented mammalian skin comprising a step of contacting the skin with a formulation comprising a composition of claim 1 in a sufficient amount to prevent excessive darkening of the skin.

30. A method of preparing a skin-lightening composition having an enhanced ability to penetrate mammalian skin and lighten a color of mammalian skin comprising dissolving about 0.01% to about 30%, by weight of a hydroxycinnamic acid, a methoxycinnamic acid, or mixtures thereof, in a compound having one or more hydroxy groups, a silicone fluid, or mixtures thereof, to form a solution, then either (a) adding the solution to a skin care formulation or (b) adding a skin care formulation to the solution or (c) admixing the solution with ingredients of a skin care formulation to provide the skin-lightening composition.

31. The method of claim 30 wherein the compound having one or more hydroxy groups comprises a monoC₁₋₄alkyl ether of an ethylene glycol oligomer, a monoC₁₋₄alkyl ether of a propylene glycol oligomer, or mixtures thereof.

32. The composition of claim 12 wherein the compound having one or more hydroxy groups is selected from the group consisting of PEG-4, diethylene glycol, triethylene glycol, and mixtures thereof.
